(19) United States
(12) Patent Application Publication
(10) Pub. No.: US 2014/0271497 A1
(43) Pub. Date: Sep. 18, 2014

(54) TREATMENT FOR DRY MOUTH EMPLOYING ORAL SAFE OXYGENATORS

(71) Applicant: Susan Morgan, San Francisco, CA (US)

(72) Inventor: Susan Morgan, San Francisco, CA (US)

(21) Appl. No.: 13/843,269

(22) Filed: Mar. 15, 2013

(51) Int. Cl. A61K 8/42; A61K 8/92; A61Q 11/00; A61K 8/22

(52) U.S. Cl. CPC A61K 8/42; A61K 8/22; A61K 8/922; A61Q 11/00
USPC 424/50; 424/53; 424/58

(57) ABSTRACT

A treatment for xerostomia, employing a composition having the active ingredient of safe oral oxygenator and a seed oil is described. The safe oral oxygenator can include either carbamide peroxide or sodium perborate and the seed oil can be macadamia nut oil. The oral care composition may further include one or more additives, such as glycerin (or another humectant), saliva enzyme, proanthocyanidins (e.g., PYCNOGENOL), resveratrol, a sugar alcohol, vitamin B₆, vitamin B₁₂, Coenzyme Q₁₀, and folic acid. The oral care composition can be used for the treatment of xerostomia by applying the oral composition to the oral cavity. In addition, this oral care composition can be used for the treatment of vocal cord dysfunction, throat closure, candida, and can be further used in women's intimate care.

### BACKGROUND

1. Field

Embodiments of the invention relate to the field of chemical composition treatments; and more specifically, to compositions including oxygenators for the treatment of xerostomia and other ailments.

2. Background

Saliva is a complex mixture of fluids, proteins, enzymes, and electrolytes that are produced by the salivary glands which surround the oral cavity, and is secreted into the mouth following appropriate stimulation of oral tissues. Secretion of saliva is regulated by the body's autonomic nervous system, which permits involuntary secretion following an appropriate stimulus such as tasting or chewing food, smelling or thinking of food, or having any object within the mouth, which stimulates the oral mucosa. A healthy saliva system also continually protects against deadly bacteria invading the oral cavity and other parts of the body.

Xerostomia occurs when inadequate amounts of saliva are secreted into the mouth, preventing adequate lubrication of the oral cavity and resulting in an uncomfortable oral sensation and difficulty with speaking and swallowing. Severe cracking of the tongue can also result. In addition to the primary role of lubricating the oral cavity during conversation and eating, saliva also limits growth of bacteria that can cause tooth decay and oral infections, promotes digestion of food, and acts as a protective barrier against demineralization of tooth enamel.

Xerostomia can result from either decreased production of saliva within the glands and/or diminished secretion of saliva from the glands following autonomic stimulation. It is most commonly caused as an unwanted side effect of many classes of prescription medications including anticholinergics, antispasmodics, antihypertensives, antidepressants, anticonvulsants, pain killers, anti-rejection drugs, and antipsychotics, as well as over-the-counter decongestants and antihistamines. These classes of drugs can either directly inhibit saliva production within the glands or inhibit its secretion into the mouth by inhibiting the autonomic nervous system.

Xerostomia can also occur during states of elevated stress, anxiety, depression, with certain endocrine diseases such as hypothyroidism, during chemotherapy, and with auto-immune disorders such as Sjogren's syndrome and lupus. Additionally, people who have had radiation therapy to the neck, traumatic injury to the neck, or neck surgery may also develop xerostomia due to destruction of the glands by the therapeutic radiation dose, or direct injury of the gland and its controlling autonomic nerves. Xerostomia is increasingly common among people of advancing age.

Typical treatment considerations for xerostomia have involved supportive and replacement therapies to restore oral moisture, as well as pharmacologic agents to stimulate the body's own saliva production.

Supportive therapy incorporates behavior and dietary modifications to keep the mouth moist throughout the day to minimize discomfort. Such strategies include frequent drinks of water, avoiding liquids with caffeine or sugar, avoiding alcohol and tobacco products, avoiding spicy or acidic foods, and following a regular daily dental hygiene program.

Replacement therapy incorporates oral administration of exogenous saliva-like compounds in order to replenish lost moisture, fluids and enzymes in the oral cavity, while introducing an appropriately viscous compound that aids in the mechanics of speaking and swallowing. Reference to current medical literature reveals several saliva replacement products.

BIOTENE, produced by GlaxoSmithKline, Inc., is a salivary enzyme replacement system in a toothpaste form that contains the active ingredient found in toothpaste (sodium monofluorophosphate) and the major enzymes found in saliva (lactoperoxidase, thiocyanate and glucoseoxidase). BIOTENE primarily functions to prevent the damage caused by harmful bacteria that can accumulate in the mouth when inadequate amounts of saliva are present. In toothpaste form, BIOTENE is less effective at long-term relief of the classic symptoms of dry mouth but is also available in mouthwash, chewing gum, and oral gel (ORALBALANCE) preparations which include oral moisturizing agents that are more effective in longer duration of symptom relief.

SALIX, produced by Scandinavian Formulas, is available in tablet form and contains sorbitol, polyethylene glycol, malic acid, hydrogenated cottonseed oil, sodium citrate, dicalcium phosphate, citric acid, silicon dioxide, and carboxy methylcellulose. It acts as a temporary semi-viscous saliva substitute to improve the mechanics of speaking and swallowing but does little to inhibit oral bacteria responsible for tooth decay.

SALAGEN, commonly known as pilocarpine hydrochloride, produced by MGI Pharma, Inc., is a pharmacologic agent that is indicated and approved for treatment of xerostomia in persons who have undergone radiation therapy to the neck and in persons with Sjogren's syndrome. Available in tablet form, SALAGEN is a cholinergic agent that is effective in pharmacologically increasing secretions from the salivary glands thereby improving the symptoms of xerostomia. SALAGEN however acts on all exocrine glands in the body and also increases secretions from the skin, eyes, pancreas, intestines, and lungs with unwanted and sometimes deleterious side-effects. Additionally, pilocarpine can cause decreased visual acuity and cardiovascular collapse with serious and possibly life-threatening consequences.

It is evident that current supportive and replacement therapies for treating xerostomia (BIOTENE and SALIX) are temporary and do little to directly stimulate the body to produce and secrete more natural saliva. Current pharmacologic therapies (SALAGEN) which do increase the body's natural saliva production also cause unwanted increased secretions in other organs and can have serious side-effects. The current invention describes a compound that relieves the symptoms of xerostomia by causing the salivary glands to produce and secrete natural saliva into the oral cavity without the serious and lifestyle-limiting side effects of current pharmacologic agents.

While the previously discussed therapies are suitable for their particular purposes, they are not suitable for the purposes of the current invention which employs an over-the-counter drug compound to increase natural saliva production and secretion in persons with xerostomia without the serious side-effects associated with currently available prescription medication.

### SUMMARY OF THE DESCRIPTION

In one embodiment, a system, which effectively treats xerostomia, and relieves the discomfort associated therewith is described. Accordingly, the current invention relates to the use of carbamide peroxide as a salivary replacement system to stimulate the body's own production of saliva by an oxygen-releasing biochemical foaming process within the mouth. In addition, the composition according to the present invention can act as a oral moisturizer and a saliva substitute.

In one embodiment, a system, which is known to be safe, so that it is approved for use in the manner of the present invention is described. Accordingly, in this embodiment, the active ingredient of the invention is carbamide peroxide. Carbamide peroxide is a stable organic compound of urea and hydrogen peroxide, which is currently employed in several over-the-counter drug remedies for removal of ear wax (MURINE, DEBROX), oral debriding and cleansing agents (GLYOXIDE, ORAJEL) and tooth whitening agents (PLATINUM OVERNIGHT). Carbamide peroxide has been fully described in FDA drug monographs listed in the Federal Register (21 CFR Parts 201, 344, 353, 356, and 369) for over-the-counter agents.

In one embodiment, a system, which is both capable of stimulating saliva production, but is also capable of clearing some mechanical blocking of the salivary ducts is described. Accordingly, the effervescent reaction of the carbamide peroxide within the mouth is capable of clearing distal blockages of the salivary ducts.

In one embodiment, a composition is used as a treatment for xerostomia, where the composition has the active ingredient of carbamide peroxide. The carbamide peroxide compound contains urea and hydrogen peroxide. The hydrogen peroxide remains stable until the carbamide peroxide is applied to the mouth and contacts the water inherent therein. On contact, the hydrogen peroxide is freed from the urea, allowing the hydrogen peroxide to react within the mouth. The resulting chemical and mechanical reaction stimulates saliva within the mouth and thereby relieves the symptoms of xerostomia.

In another embodiment, the composition includes any type of oral safe oxygenator, including a carbamide peroxide, sodium perborate, or another oral safe oxygenator as known in the art. In a further embodiment, the composition can further include a seed oil, such as macadamia nut oil, olive oil, or another type seed oil. In one embodiment, the seed oil can act as a moisturizer or an anti-inflammatory.

In another embodiment, the composition further includes one or more additional additives, where each additive is a saliva enzyme, proanthocyanidins (e.g., PYCNOGENOL), resveratrol, a sugar alcohol, vitamin B₆, vitamin B₁₂, Coenzyme Q₁₀, and folic acid. The saliva enzyme can be one or more of lactoperoxidase, thiocyanate, glucoseoxidase, and/or another type of saliva enzyme. The sugar alcohol can be xylitol or another sugar alcohol.

In a further embodiment, the oral care composition can be in a toothpaste, dental floss, tooth powder, oral spray, gum, oral strip, oral gel, oral foam, lozenge, liquid teeth cleaner, mouth rinse, and mouthwash.

In another embodiment, the oral care composition can be used to treat other ailments, such as vocal cord dysfunction, throat closure, and candida. In addition, the oral care composition can be used for women's intimate care.

To the accomplishment of the above and related objects the invention may be embodied in the form described in the accompanying specification. Attention is called to the fact, however, that the examples given are illustrative only. Variations are contemplated as being part of the invention, limited only by the scope of the claims.

### DETAILED DESCRIPTION

In one embodiment, a composition having the active ingredient of a safe oral oxygenator is employed. Hydrogen peroxide is a powerful oxidizing agent and has been used extensively in wound cleaning applications. It is a mild irritant to mucous membranes such as oral tissues. However, for the purposes of the present invention, hydrogen peroxide by itself is too unstable. If applied directly to the mouth, it quickly and violently reacts, but ceases its action within a very short period of time. Ironically, when attempting to use ordinary hydrogen peroxide for the purposes the present invention, the mouth is left with a dry feeling.

However, when hydrogen peroxide is combined with urea in the form of carbamide peroxide, it remains in a stable form until contact with skin tissues and mucous membranes. Upon contact with skin and tissues carbamide peroxide releases hydrogen peroxide, which actively destroys harmful bacteria within the mouth, removes decaying tissues and most importantly, stimulates saliva secretion. In addition, exuberant amounts of oxygen are released in a mechanical foaming action as part of the biochemical conversion of hydrogen peroxide to water and oxygen. Furthermore, this “foaming action” acts as a further mechanical stimulant to salivary gland stimulation and saliva secretion. Both the chemical and mechanical actions of carbamide peroxide stimulating the salivary glands apparently serve to reduce or eliminate the effects of xerostomia.

Because the hydrogen peroxide remains stable until it is released from the urea, the carbamide peroxide in effect becomes a “time releasing” agent for the hydrogen peroxide, and seems to continue releasing low levels of hydrogen peroxide for at least twelve hours. Once applied to the mouth, the carbamide peroxide can be effective at reducing or eliminating xerostomia for several days. It is believed that the delayed or indirect release of hydrogen peroxide provides the effective longevity of the carbamide peroxide for the purposes of the present invention.

### Carbamide Peroxide Composition

The present invention can further serve to enhance proper saliva release by clearing mechanical blockages or “stones” within the salivary ducts. The “foaming action” can serve to clear stones within the salivary ducts, but is especially effective at clearing distal stones and other forms of distal blockages.

Use of the present invention involves the oral application of the composition to a patient suffering from xerostomia. Immediately upon contact with skin tissues and mucous membranes, water inherent therein begins causing urea from the carbamide peroxide to split from the hydrogen peroxide. The hydrogen peroxide then causes a strongly mechanical reaction wherein oxygen is rapidly released, and minor irritation to oral tissues results. The reaction also causes stimulation of the salivary glands and subsequent relief from the symptoms of xerostomia.

In one embodiment, the composition is a mixture of 10 percent carbamide peroxide in glycerin. Of course, different strengths may be employed, the most suitable of which may be determined by empirical testing. Keeping the hydrogen peroxide stably within the carbamide peroxide compound prior to use involves keeping the carbamide peroxide isolated from water. Accordingly, the carbamide peroxide may be maintained in a glycerin suspension. The glycerin can act as a moisturizer to further aid the invention in its goal of being an oral moisturizer.

In one embodiment, the composition is applied directly to the mouth of a person suffering from xerostomia using an applicator brush, such as a toothbrush. The composition is applied directly to the brush, and is preferably not mixed with toothpaste or any other substance. Once on the brush, the composition is applied to all interior surfaces of the mouth—the roof of the mouth, the tongue, under the tongue, the gums, the inside of the cheeks, etc. As all surfaces of the mouth are coated, the hydrogen peroxide is released, causing a chemical reaction to immediately commence. The effervescent action of the hydrogen peroxide is most pronounced immediately following application, but then apparently dies down to a slow, time-released reaction. It seems that the continuing reaction keeps the dry-mouth curing properties of the invention working for a considerable period of time. By the preferred manner of usage of the invention, the application process is completed approximately twice daily (every nine to twelve hours). However, one application of the carbamide peroxide composition can be effective at preventing dry mouth for several days.

### Oral Care Composition Using a Safe Oral Oxygenator

As described above, a carbamide peroxide glycerin composition is used for treatment of xerostomia. In this composition, the glycerin protects the carbamide peroxide from water and the hydrogen peroxide is released from the composition upon contact with water in the patient's mouth. In one embodiment, the released hydrogen peroxide causes a strongly mechanical reaction where oxygen is rapidly released, and minor irritation to oral tissues results. The reaction also causes stimulation of the salivary glands and subsequent relief from the symptoms of xerostomia.

While the above composition used carbamide peroxide, any type of safe oral oxygenator can be used in a oral care composition to treat xerostomia. The oxygen laden foam in safe oral oxygenators can kill germs over a long period. In addition, these safe oral oxygenators destroy the volatile sulfur compounds on the tongue while also stimulating saliva and providing other benefits of healthy saliva (e.g. fresh, naturally sweet breath; lack of sticky mucous; and/or saliva glands that are kept free of debris). The use of a safe oral oxygenator allows the properties of the xerostomia treatment to last for a prolonged time. In one embodiment, an oral care composition includes sodium perborate. In one embodiment, sodium perborate is a white, free-flowing crystalline compound soluble in water and is a stable, solid source of active oxygen. Sodium perborate exists in the anhydrous, mono, tri and tetrahydrate forms. In one embodiment, the safe oxygenator is sodium perborate monohydrate or sodium perborate tetrahydrate, both of which are safe for use in the oral cavity. Sodium perborate tetrahydrate is prepared by reaction of sodium borate with hydrogen peroxide. In one embodiment, sodium perborate releases hydrogen peroxide, if contacted with water. In this embodiment, sodium perborate is considered as a solid form of hydrogen peroxide and can be used as a strong oxidizing agent. Sodium perborate monohydrate provides a high available oxygen, and thus, sodium perborate is an oxygenator that releases oxygen upon contact with water. In one embodiment, the sodium perborate is mixed with a humectant, such as glycerin. In this embodiment, the glycerin maintains the sodium perborate until the mixture is applied to the oral cavity. Sodium perborate monohydrate is permitted in countries where carbamide peroxide is not permitted over the counter. In one embodiment, the composition is a mixture of 5-15% sodium perborate in glycerin. For example and in one embodiment, the composition is 10% sodium perborate in glycerin.

In one embodiment, the oral care composition can include one or more ingredients beside the safe oral oxygenator. For example and in one embodiment, the oral care composition can include a seed oil, such as macadamia nut oil, olive oil, or another type of seed oil. These seed oils can act as anti-inflamatory and as a humectant. In one embodiment, the seed oil is macadamia nut oil, which is a non-volatile oil expressed from the nut meat of the macadamia. Macadamia nut oil is heat resistant, as the smoke point is 210° C. In this embodiment, macadamia nut oil may act as a humectant but also acts as an anti-inflammatory. Macadamia nut oil may additionally have other beneficial use for the human body. Furthermore, macadamia nut oil can act as a moisturizer and has a pleasant nutty sweet taste, which each can aid in the use of the oral care composition. In one embodiment, the macadamia nut oil is oil made in Australia and is produced in a facility that is not used to produce other nuts or peanuts. This is done to avoid cross-contamination with peanuts or other types of nuts, which can cause allergies in some people. In another embodiment, olive oil can be used in the oral care composition, either with or without the macadamia nut oil. In one embodiment, any healthy seed oil can be used (e.g., sunflower oil) in the oral care composition. Sunflower oil repairs body, promotes healthy immune system. In addition, this oil helps create new cells and contains zinc which helps maintain a healthy immune system.

In one embodiment, the oral care composition may further include one or more other additives. These additives can be used for a variety of reasons to supplement the safe oral oxygenators and/or seed oil of the oral care composition. In one embodiment, the additives can be one or more of each additive is proanthocyanidins, resveratrol, a saliva enzyme, a sugar alcohol, vitamin B₁₂, Coenzyme Q₁₀ (e.g., ubiquinol), potassium nitrate (or another known tooth desensitizer), and folic acid. The saliva enzyme can be one or more of lactoperoxidase, thiocyanate, glucoseoxidase, and/or another type of saliva enzyme. The sugar alcohol can be xylitol or another sugar alcohol. In one embodiment, resveratrol can improve cell renewal, which may repair damaged saliva glands. In one embodiment, vitamin B₆ is helpful to reduce stress, which can lead to xerostomia. In another embodiment, Coenzyme Q₁₀ is used in the oral care composition as Coenzyme Q₁₀ can act as a saliva stimulant. In addition, some of these additives have anti-oxidant properties, which is beneficial to a patient's health.

In one embodiment, the oral care composition includes proanthocyanidins that is an abundant condensed tannin. In one embodiment, the proanthocyanidins is an extract from the tree bark of French maritime pine trees (e.g., PYCNOGENOL). In one embodiment, PYCNOGENOL strengthens capillaries and blood vessels to improve circulation and clearing out unhealthy matter in the oral cavity. For example, PYCNOGENOL, can reduce plaque build-up in the capillaries and/or blood vessels in the oral cavity, which strengthens capillaries in the saliva glands. In particular, the proanthocyanidinextracted from the tree bark of French maritime pine trees has been shown to clear capillaries, which should keep capillaries in the oral cavity clear of sticky plaque, blood sludge, clots, etc. Furthermore, the addition of the proanthocyanidins to the oral care composition could benefit those with autoimmune diseases including lupus and Sjogren's disease where dryness of the body may include low to no salivary function. Also, patients that have received chemotherapy and/or radiation therapy often suffer from damaged or destroyed saliva glands. In addition, the proanthocyanidins have been shown to rebuild collagen and elastin. In one embodiment, the amount of proanthocyanidins included in an application of the oral care composition ranges from 15-50 mg. In one embodiment, another type of proanthocyanidins that can be used in the oral care composition is grape seed extract, which has similar properties to PYCNOGENOL (e.g., building and protecting collagen). In addition, grape seed extract provides another type of proanthocyanidin not found in pine bark extract (e.g., B2-3-O-gallate).

In one embodiment, another of the additives is a saliva enzyme. As described above the saliva enzyme replacement system, BIOTENE, includes saliva enzymes. In this embodiment, one or more of saliva enzymes is included in the oral care composition. For example and in one embodiment, lactoperoxidase, thiocyanate and/or glucoseoxidase can be included in the oral care composition. In one embodiment, the oral care composition includes an oral safe oxygenator, a seed oil, and one or more of the saliva enzymes. In another embodiment, the oral care composition includes a safe oral oxygenator and the active ingredients of BIOTENE (e.g., sodium monofluorophosphate, lactoperoxidase, glucoseoxidase, lactoferrin, lysozyme, and/or other active ingredients of BIOTENE). In this embodiment, a seed oil (e.g., macadamia nut oil), proanthocyanidins extracted from the tree bark of French maritime pine trees (e.g., PYCNOGENOL), and/or a humectant may also be included (e.g., glycerin). In a further embodiment, the active ingredients of one or more of the BIOXTRA products can be included in the oral care composition. For example and in one embodiment, the oral care composition can include a safe oral oxygenator, a seed oil, the active ingredients of one or more of the BIOXTRA products, proanthocyanidins extracted from the tree bark of French maritime pine trees (e.g., PYCNOGENOL), a humectant, and/or a combination therein. In a further embodiment, the oral care composition can include any other type of saliva stimulant, saliva substitute, and/or saliva substitute.

In another embodiment, a sugar alcohol may be added to the oral care composition. In one embodiment, the sugar alcohol is xylitol, which is an oral moisturizer and a saliva stimulant. In this embodiment, xylitol is also a sweetener.

In one embodiment, the oral care composition is applied directly to the mouth of a person suffering from xerostomia using an applicator brush, such as a toothbrush. The composition is applied directly to the brush. Once on the brush, the composition is applied to the interior surfaces of the mouth—the roof of the mouth, the tongue, under the tongue, the gums, the inside of the cheeks, etc. As the surfaces of the mouth are coated, the oxygenator provides the oxygen to stimulate saliva. For example and in one embodiment, if the oxygenator is carbamide peroxide, the hydrogen peroxide is released, causing a chemical reaction to immediately commence. The effervescent action of the hydrogen peroxide is most pronounced immediately following application, but then apparently dies down to a slow, time-released reaction. It seems that the continuing reaction keeps the dry-mouth curing properties of the invention working for a considerable period of time. By the preferred manner of usage of the invention, the application process is completed approximately twice daily (every six to twelve hours). However, one application of the carbamide peroxide composition can be effective at preventing dry mouth for several days.

In one embodiment, the oral care composition can be used in a variety of forms. For example and in one embodiment, the oral care composition can be used in toothpaste, other types of oral paste, dental floss, tooth powder, oral spray, gum, oral strip, oral gel, oral foam, lozenge, liquid teeth cleaner, mouth rinse, and mouthwash. In one embodiment, the oral care composition can be used as a toothpaste, in conjugation with a toothpaste and/or as a toothpaste finish. When used as a toothpaste finish, the patient brushes the teeth and applies the oral care composition after brushing. In another embodiment, dental floss is impregnated or covered with the oral care composition, in which the patient uses the dental floss to apply to oral care composition to the gums and other parts of the oral cavity. In another embodiment, the oral care composition is applied using a toothbrush, swab, or other type of applicator. In a further embodiment, a two chamber system can be used to deliver the oral care composition, where one chamber includes some of the components of the oral care composition (e.g., the safe oral oxygenator), and the other chamber include the rest of the oral care components (e.g., macadamia seed oil).

### Alternative Treatments Using Oxygenator-Based Compositions

As described above, the oxygenator-based oral care compositions can be used to treat xerostomia because these compositions can stimulate saliva, act as an anti-inflammatory, and moisturize the surface of the oral cavity. These properties allow the oral care composition to be used as a treatment for other physical ailments. In one embodiment, this oral care composition can be used to treat vocal cord dysfunction, throat closure and candida. In one embodiment, the oral care composition helps a lupus patient by acting as a moisturizer of the oral cavity if the salivary glands are damaged, destroyed, or otherwise non-functional.

In one embodiment, vocal cord dysfunction is a condition that affects the vocal folds, commonly referred to as the vocal cords, and is characterized by full or partial vocal fold closure that can occurs during inhalation and/or exhalation for short periods of time. This closure may cause airflow obstruction. Vocal cord dysfunction can be caused by many different factors, such as gastroesophageal reflux disease, extra-esophageal reflux, exposure to inhaled allergens, post nasal drip, exercise, or neurological conditions that can cause difficulty inhaling only during waking. Also certain medications, such as antihistamines for allergies, can provide a drying effect to the mucus membranes, which can further cause the vocal cords to be irritated or hypersensitive. Vocal cords dysfunction may also be a side effect of drugs including some steroid inhalers. Vocal cords dysfunction may also be caused by dry mouth including patients that have received chemo or radiation treatment of the head and neck.

The saliva stimulation and anti-inflammatory properties of the oral care composition can be used to treat a patient with vocal cord dysfunction. In one embodiment, the anti-inflammatory properties of the oral care composition can be used to treat vocal cord dysfunction and/or throat closure by reducing the inflammation that is caused by these ailments. In one embodiment, the addition of PYCNOGENOL to the oral care composition helps strengthen the capillaries and collagen of the oral cavity. This, in turn, improves the salivary system which allows healthy saliva to moisturize the throat area. With the blood and saliva flowing freely in the affected area of the throat, the problems from vocal cord dysfunction and throat closure are reduced, because the saliva system is enhanced. In one embodiment, a treatment for vocal cord dysfunction and/or throat closure can be performed using any one of the ways described above for xerostomia treatment.

In one embodiment, candida (also known as oral thrush) is treated by stimulating saliva using any one of the oral care compositions described above. This treatment kills deadly bacteria in the oral cavity as well as destroys volatile sulfur compounds. In one embodiment, a treatment for vocal cord dysfunction and/or throat closure can be performed using any one of the ways described above for xerostomia treatment. In one embodiment, candida is potentially fatal and the treatment of candida using the oral care composition allows a candida patient to be treated with medication.

In addition to treating xerostomia and other ailments, the oral care composition can be used for intimate care for men and women. In one embodiment, the oral care composition can be used for intimate care of premenopausal, menopausal, and/or post-menopausal women who suffer from vaginal dryness. This tissue is similar to the oral cavity and therefore, moisture, even natural moisture, could be stimulated using the oral care composition. In one embodiment, the oral care composition for women's intimate use comprises a safe oxygenator (e.g., carbamide peroxide or a form of sodium perborate), and macadamia nut oil. In another embodiment, this oral care composition can further include glycerin and/or one or more of the additives described above (e.g., resveratrol, PYCNOGENOL, Coenzyme Q₁₀, etc.). In this embodiment, the oral care composition can repair collagen elastin and further stimulate moisture. In one embodiment, the oral care composition can be put into a cream, gel, or other type of treatment that is used for women's intimate care. This can be applied using a type of applicator used in women's intimate care. In one embodiment, the oral care composition can further treat yeast infection.

The foregoing description of the system and the reasons for its effectiveness is presented as best known by the inventor at the time of application. Although the precise mechanism of the invention and the reasons for its effectiveness will be determined in greater detail at a later date following further study, the invention is not limited by those embodiments which are presently known and described herein. Variations of the invention, using the same principles of the present invention in light of future discoveries of the properties of the present invention and suitable chemical substitutions therefor are contemplated as being part of the present invention, limited only by the scope of the claims.

What is claimed is:

1. An oral care composition, the composition comprising:
a safe oral oxygenator; and
a seed oil.

2. The oral care composition of claim 1, wherein the safe oral oxygenator is selected from the group consisting of carbamide peroxide, sodium perborate mono hydrate, and sodium perborate tetrahydrate.

3. The oral care composition of claim 1, wherein the seed oil is selected from the group consisting of macadamia nut oil, olive oil, and sunflower oil.

4. The oral care composition of claim 1, further comprising:
a humectant carrier.

5. The oral care composition of claim 4, wherein the humectant carrier is glycerin.

6. The oral care composition of claim 4, wherein the percentage of the safe oral oxygenator is 5-15% (w/v).

7. The oral care composition of claim 6, wherein the percentage of the safe oral oxygenator is 10% (w/v).

8. The oral care composition of claim 1, further comprising:
an additive, wherein the additive is selected from the group consisting of a saliva enzyme, proanthocyanidins, resveratrol, a sugar alcohol, vitamin B₆, vitamin B₁₂, Q₁₀, and folic acid.

9. The oral care composition of claim 8, wherein the proanthocyanidins is PYCNOGENOL.

10. The oral care composition of claim 8, wherein the sugar alcohol is xylitol.

11. The oral care composition of claim 8, wherein the saliva enzyme is selected from the group consisting of lactoperoxidase, thiocyanate and glucoseoxidase.

12. The oral care composition of claim 1, wherein the oral care composition is in a delivery system, wherein the delivery system is selected from the group consisting of toothpaste, gums, foams, strips, gels, liquid teeth cleaners, mouth rinses, and mouthwash.

13. A method of treating xerostomia, comprising:
providing an oral care composition that includes a safe oral oxygenator and a seed oil; and
applying the oral care composition to interior surfaces of the mouth of a patient suffering from xerostomia, wherein the oral care composition releases hydrogen peroxide upon contact with the interior surfaces of the mouth.

14. The method of claim 13, further comprising:
repeatedly applying the composition to the interior surfaces of the mouth of the patient substantially every six to twelve hours.

15. The method of claim 14, wherein the applying the composition to interior surfaces of the mouth further comprises the steps of:
applying the composition to an applicator; and
brushing the interior surfaces of the mouth with the applicator.

16. The method of claim 13, wherein the safe oral oxygenator is selected from the group consisting of carbamide peroxide and sodium perborate.

17. The method of claim 13, wherein the seed oil is selected from the group consisting of macadamia nut oil, olive oil, and sunflower oil.

18. The method of claim 13, wherein the oral care composition further comprises:
an additive, wherein the additive is selected from the group consisting of a saliva enzyme, proanthocyanidins, resveratrol, a sugar alcohol, vitamin B₁₂, Coenzyme Q₁₀, and folic acid.

19. The method of claim 18, wherein the sugar alcohol is xylitol.

20. The method of claim 13, wherein the oral care composition is in a delivery system, wherein the delivery system is selected from the group consisting of toothpaste, gums, strips, foams, gels, liquid teeth cleaners, mouth rinses, and mouthwash.
